(19) United States
(12) Patent Application Publication
(10) Pub. No.: US 2009/0075306 A1
(43) Pub. Date: Mar. 19, 2009

(54) DIAGNOSTIC ASSAY FOR DETECTING AND MONITORING AGE RELATED AND NOISE INDUCED HEARING LOSS

(75) Inventors: Vincent K. Tuohy, Broadview Heights, OH (US); Gordon B. Hughes, Chagrin Falls, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/116,555

(22) Filed: May 7, 2008

Related U.S. Application Data
(63) Continuation-in-part of application No. PCT/US2007/077686, filed on Sep. 7, 2006.
(60) Provisional application No. 60/842,923, filed on Sep. 7, 2006.

(51) Int. Cl. G01N 33/53; G01N 33/564

(52) U.S. Cl. 435/7.92; 436/506

(57) ABSTRACT

A method of detecting age related and/or noise induced hearing loss in a subject includes detecting in a biological sample from the subject the presence of cochlin antibodies; and correlating the level of detected cochlin antibodies to the presence or absence of age related and/or noise induced hearing loss in the subject.

### RELATED APPLICATION

This application is a Continuation-in-Part of Int'l Application No. PCT/US2007/077686, filed Sep. 6, 2007, which claims priority from U.S. Provisional Application No. 60/842,923, filed Sep. 7, 2006, the subject matter, which is incorporated herein by reference.

### GOVERNMENT FUNDING

This invention was made with government support under Grant No. NIH/NIDCD R01-DC003402 awarded by the National Institutes of Health. The government has certain rights in the invention.

### FIELD OF THE INVENTION

The present invention relates generally to the detection of age related and/or noise induced hearing loss in a subject, and more specifically to an immunoassay for the diagnosis of age related and/or noise induced hearing loss.

### BACKGROUND

Autoimmune sensorineural hearing loss (ASNHL) is thought to be caused by autoimmune responses against inner-ear specific differentiation proteins. ASNHL is a form of sensorineural hearing loss caused by a malfunction of the body's immune system, which attacks and progressively destroys the inner ear. The pathogenesis of ASNHL may include vasculitis of vessels supplying the inner ear, autoantibodies directed against inner ear antigenic epitopes or cross-reacting antibodies. ASNHL is characterized by progressive unilateral or bilateral deafness that, in its incipient stages, may fluctuate or become sudden and profound. The symptoms of ASNHL are quite similar to other forms of sensorineural hearing loss (SNHL). Although autoimmune etiopathogenic events have long been identified in ASNHL, inner ear specific antigens capable of targeting T cell autoimmunity had yet to be identified in ASNHL.

### SUMMARY

The present invention relates to a method of detecting age related and/or noise induced hearing loss in a subject. The method includes detecting in a biological sample from the subject the presence of cochlin antibodies. The level of detected cochlin antibodies is correlated to the presence or absence of age related and/or noise induced hearing loss in the subject. The detection of an increased level of cochlin antibodies in the biological sample compared to the level of cochlin antibodies in a control population of subjects that do not have age related and/or noise induced hearing loss indicates presence of or increased risk of age related and/or noise induced hearing loss in the subject.

In an aspect of the invention, the cochlin antibodies can be detected by an immunological method, such as an ELISA. In the immunological method, the biological sample is contacted with cochlin. Cochlin antibodies bound to the cochlin are then contacted with detectable antibodies. The level of the detectable antibodies bound to the cochlin antibodies are subsequently determined. The level of bound detectable antibodies corresponds to the level of cochlin antibodies in the biological sample.

In a further aspect, the cochlin can be immobilized on a solid support, such as a 96 well microtiter plate. The biological sample can include blood plasma, serum, or whole blood from the subject. The cochlin immobilized on the solid support can include recombinant human cochlin.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a schematic flow diagram illustrating a method in accordance with an aspect of the present invention.

FIG. 2 is a graph depicting sera from ASNHL patients showing elevated cochlin antibody titers compared to control subjects.

### DETAILED DESCRIPTION

The present invention relates to a diagnostic method of detecting age related and/or noise induced hearing loss in a subject and also to a method of monitoring the effect of therapies directed to treating age related and/or noise induced hearing loss in the subject. Subjects with age related and/or noise induced hearing loss have increased circulating blood or sera levels of cochlin antibodies compared to normal or healthy subjects that do not have age related and/or noise induced hearing loss and in some instances can have decreased levels of cochlin antibodies compared to subjects with autoimmune sensorineural hearing loss (ASNHL). The detection and/or determination of a subject's cochlin antibody levels in biological samples obtained from the subject can therefore be used in diagnostic methods of the present invention including detecting and/or characterizing noise induced hearing loss, detecting and/or characterizing age related hearing loss, detecting and/or characterizing inner ear damage that may lead to progressively deteriorating hearing if behavior change is not instituted (e.g., inner ear damage associated with work place environment), monitoring an age related and/or noise induced hearing loss subject's response to a therapeutic treatment, and kits for assaying for the presence of cochlin antibodies associated with age related and/or noise induced hearing loss in a subject.

The term “biological sample” refers to a body sample from any animal, but preferably is from a mammal, more preferably from a human. Such samples include biological fluids, such as serum, plasma, vitreous fluid, lymph fluid, synovial fluid, follicular fluid, seminal fluid, amniotic fluid, milk, whole blood and tissue culture medium, as well as tissue extracts, such as homogenized tissue, and cellular extracts.

The term “detecting” in accordance with the present invention is used in the broadest sense to include both qualitative and quantitative measurements of cochlin antibody. In one aspect, the detecting method as described herein is used to identify the mere presence of cochlin antibody in a biological sample. In another aspect, the method can be used to quantify the amount or level of cochlin antibody in a biological sample and further to compare the amount of cochlin antibody in different samples. For example, the different samples can be biological samples from individuals that do not have age related, noise induced, and/or autoimmune sensorineural hearing loss.

The term “detecting-antibody”, “detectable antibody”, or “detection antibody” refers to an antibody that is capable of being detected, for example, either directly through a label amplified by a detection means, or indirectly through, e.g., another antibody that is labeled. For direct labeling, the antibody is typically conjugated to a moiety that is detectable by some means.

The term “detection means” refers to a moiety or technique used to detect the presence of the detectable antibody and can include detection agents that amplify the immobilized label such as label captured onto a microtiter plate.

The term “antibody” is used in the broadest sense and includes monoclonal antibodies (including agonist, antagonist, and neutralizing antibodies) and epitope binding antibody fragments thereof so long as they exhibit the desired binding specificity.

The term “monoclonal antibody” as used herein refers to an antibody obtained from a population of homogeneous antibodies, i.e., the individual antibodies comprising the population are identical except for possible naturally-occurring mutations that may be present in minor amounts. Monoclonal antibodies are highly specific, being directed against a single epitope binding site. Furthermore, in contrast to conventional (polyclonal) antibody preparations that typically include different antibodies directed against different determinants (epitopes), each monoclonal antibody is directed against a single determinant on the protein. The modifier “monoclonal” indicates the character of the antibody as being obtained from a homogeneous population of antibodies, and is not to be construed as requiring production of the antibody by any particular method. For example, the monoclonal antibodies to be used in accordance with the present invention may be made by the hybridoma method first described by Kohler et al. Nature 256:495 (1975), or may be made by recombinant DNA methods (see, e.g., U.S. Pat. No. 4,816,567, herein incorporated by reference in its entirety). The “monoclonal antibodies” may also be isolated from phage antibody libraries using the techniques described in Clackson et al Nature 352:624-628 (1991) and Marks et al. J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody), such as mouse, rat, rabbit or nonhuman primate having the desired specificity, affinity, and capacity.

“Mammal” for purposes of treatment refers to any animal classified as a mammal, including humans, domestic, and farm animals, and zoo, sports, or pet animals, such as dogs, horses, cats, sheep, pigs, cows, etc. Preferably, the mammal is human.

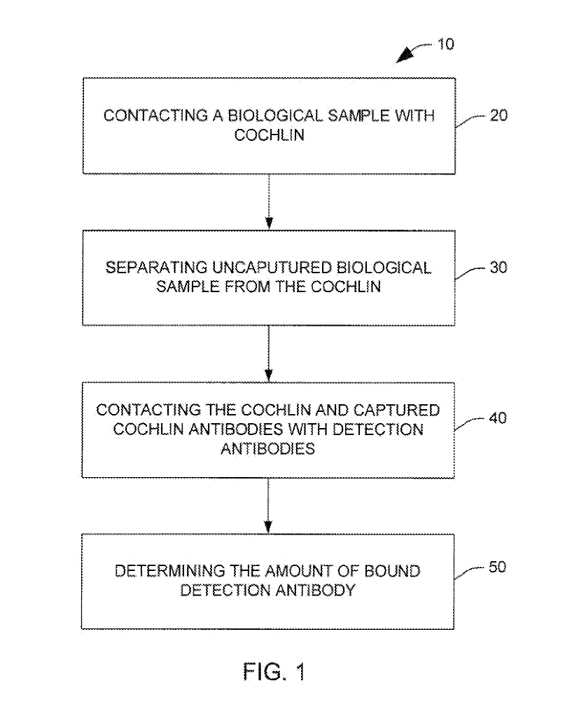

In accordance with an aspect of the present invention, the cochlin antibodies can be detected in a biological sample from a subject using an immunological detection method to determine if the subject has age related and/or noise induced hearing loss. The immunological method can include an enzyme linked immunosorbent assay (ELISA). FIG. 1 is a schematic flow diagram of an example of an ELISA detection method 10 that can be used to detect cochlin antibodies in a biological sample of a subject suspected of having age related and/or noise induced hearing loss.

In the ELISA detection method 10, at step 20, a biological sample obtained from the subject is contacted with cochlin. The cochlin can include cochlin proteins, and biologically active portions thereof, as well as peptide fragments that can bind to cochlin antibodies. An isolated or purified protein or biologically active portion thereof is substantially free of cellular material when produced by recombinant DNA techniques, or chemical precursors or other chemicals when chemically synthesized. The language “substantially free of cellular material” includes preparations of cochlin in which the protein is separated from cellular components of the cells in which it is naturally or recombinantly produced. The cochlin protein can also be substantially free of culture medium and chemical precursors or other chemicals.

The cochlin can be produced, for example, by recombinant DNA techniques. In one aspect, a human cDNA nucleic acid molecule encoding cochlin protein can be cloned into an expression vector. The expression vector can be introduced into a prokaryotic or eukaryotic host cell, and the cochlin protein can be expressed in the host cell. Examples of host cells that can be used are known to those having skill in the art. The cochlin protein can then be isolated from the cells by an appropriate purification scheme using standard protein purification techniques. Alternative to recombinant expression, a cochlin protein, polypeptide, or peptide can be synthesized chemically using standard peptide synthesis techniques. Moreover, native cochlin protein can be isolated from cells (e.g., cells of the inner ear).

The cochlin can also include a cochlin fusion protein. As used herein, a cochlin fusion protein includes a cochlin polypeptide operatively linked (e.g., fused in-frame to each other) to a non-cochlin polypeptide. For example, the fusion protein can be GST-cochlin fusion protein in which cochlin sequences are fused to the C-terminus of the GST sequences. Such fusion proteins can facilitate the purification of recombinant cochlin. In another embodiment, the fusion protein can be a cochlin protein containing a heterologous signal sequence at its N-terminus. In certain host cells (e.g., mammalian host cells), expression and/or secretion of cochlin can be increased through use of a heterologous signal sequence. Polyhistidine-tags can be utilized used for affinity purification of polyhistidine-tagged recombinant proteins that are expressed in Escherichia coli or other prokaryotic expression systems.

The cochlin can also include homologues of the cochlin proteins which function as a cochlin agonist (e.g., mimetic) and which can bind to cochlin antibodies. As used herein, the term “homologue” refers to a variant form of the cochlin protein which acts as an agonist of the activity of the cochlin protein. An agonist of the cochlin protein can retain substantially the same, or a subset of the biological activities of the cochlin protein. Homologues of the cochlin protein can be generated by mutagenesis, e.g., discrete point mutation or truncation of the cochlin protein.

The cochlin can also include immunopurified cochlin and/or can be recombinant human cochlin. Protein purification is a series of processes intended to isolate a single type of protein from a complex mixture. Protein purification is vital for the characterization of the function, structure and interactions of the protein of interest. The starting material is usually a biological tissue or a microbial culture. The various steps in the purification process may free the protein from a matrix that confines it, separate the protein and non-protein parts of the mixture, and finally separate the desired protein from all other proteins. Separation steps exploit differences in protein size, physical-chemical properties and binding affinity. Protein separation methods are well known to those who practice in the art.

In an aspect of the invention, the cochlin contacted with the biological sample can be immobilized on a solid phase. For example, the cochlin can be immobilized on a solid phase by adsorption of the cochlin to a water-insoluble matrix or surface (U.S. Pat. No. 3,720,760, herein incorporated by reference in its entirety) or by non-covalent or covalent coupling, for example, using glutaraldehyde or carbodiimide cross-linking, with or without prior activation of the surface (e.g., with nitric acid and a reducing agent as described in U.S. Pat. No. 3,645,852 or in Rotmans et al., J. Immunol. Methods 57:87-98 (1983)).

The solid phase used for immobilization may be any inert support or carrier that is essentially water insoluble and useful in immunometric assays. Such supports can be in the form of surfaces, particles, porous matrices, etc. Examples of commonly used supports include small sheets, Sephadex, polyvinyl chloride, plastic beads, and assay plates or test tubes manufactured from polyethylene, polypropylene, polystyrene, and the like including 96-well microtiter plates and 384-well microtiter well plates, as well as particulate materials, such as filter paper, agarose, cross-linked dextran, and other polysaccharides. Alternatively, reactive water-insoluble matrices, such as cyanogen bromide-activated carbohydrates and the reactive substrates described in U.S. Pat. Nos. 3,969,287; 3,691,016; 4,195,128; 4,247,642; 4,229,537; and 4,330,440 can be employed for cochlin immobilization. In one example, the cochlin is coated on a microtiter plate, such as, a multi-well microtiter plate that can be used to analyze several samples at one time. For example, the multi-well microtiter plate can be a microtest 96-well ELISA plate, such as that sold by Nune Maxisorb or Immulon.

If micro-titer well plates (e.g., 96-well plates or 384-well plates) are utilized, they can be coated with the cochlin at, for example, room temperature. The plates may be stacked and coated long in advance of the assay itself, and then the assay can be carried out simultaneously on several samples in a manual, semi-automatic, or automatic fashion, such as by using robotics.

The biological sample combined with the cochlin can potentially include cochlin antibodies to be detected. In one example, the biological sample can include a biological fluid, such as whole blood, sera, or plasma obtained from a subject. The biological sample can also be diluted with, for example, a buffer (e.g., Dulbecco's phosphate-buffered saline (DPBS) with 0.05% Tween-20 (PBT)).

For sufficient sensitivity, the amount of biological sample combined with the immobilized cochlin can be such that the immobilized cochlin is in molar excess of the maximum molar concentration of the cochlin antibodies anticipated in the biological sample after appropriate dilution of the sample. This anticipated level depends mainly on any known correlation between the concentration levels of the cochlin antibodies in the particular biological sample being analyzed with the clinical condition of the patient.

Following contact of the biological sample with the immobilized cochlin, at step 30, the biological sample is separated (e.g., by washing) from the immobilized cochlin to remove uncaptured cochlin antibodies. The solution used for washing can include a buffer (“washing buffer”) with a pH determined using the considerations and buffers typically used for the incubation step. The washing may be done, for example, three or more times. The temperature of washing is generally from refrigerator to moderate temperatures, with a constant temperature maintained during the assay period, typically from 0 to about 40° C. Optionally, a cross-linking agent or other suitable agent may be added at this stage to allow the now-bound cochlin antibodies to be covalently attached to the cochlin if there is any concern that the captured cochlin antibodies may dissociate to some extent in the subsequent steps.

Following separation of the uncaptured biological sample, at step 40, the immobilized cochlin and bound cochlin antibodies are contacted with detecting antibodies (or epitope binding fragments thereof). The detecting antibody can comprise antibodies or antibody fragments to the bound cochlin antibodies. A molar excess of the detecting antibody with respect to the maximum concentration of free binding epitopes expected is added to the bound cochlin antibodies after they are washed.

The detecting antibody can be labeled with any detectable functionality that does not interfere with the binding of the detecting antibody to free binding epitopes on the bound cochlin antibodies. Examples of labels are those numerous labels known for use in immunoassays, including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores, such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, peroxidase, horseradish peroxidase (HRP), alkaline phosphitase, β-galactosidase, glucoamylase, lysozyme, saccharide oxidases, e.g., glucose oxidase, galactose oxidase, and glucose-6-phosphate dehydrogenase, heterocyclic oxidases, such as uricase and xanthine oxidase, coupled with an enzyme that employs hydrogen peroxide to oxidize a dye precursor, such as HPP, lactoperoxidase, or microperoxidase, biotin/avidin, biotin/streptavidin, biotin/Streptavidin-β-galactosidase with MUG, spin labels, bacteriophage labels, stable free-radicals, and the like.

Conventional methods are available to bind these labels covalently to proteins or polypeptides. For instance, coupling agents, such as dialdehydes, carbodiimides, dimaleimides, bis-imidates, bis-diazotized benzidine, and the like may be used to tag the antibodies with the above-described fluorescent, chemiluminescent, and enzyme labels, e.g., U.S. Pat. Nos. 3,940,475 (fluorimetry) and 3,645,090 (enzymes); Hunter et al Nature 144:945 (1962); David et al. Biochemistry 13:1014-1021 (1974); Pain et al. J. Immunol Methods 40:219 230 (1981); and Nygren J. Histochem and Cytochem 30:407-412 (1982).

The conjugation of such label, including the enzymes, to the antibody is a standard manipulative procedure for one of ordinary skill in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Following the addition of detecting antibodies, at step 50, the amount or level of bound detecting-antibody is determined by removing excess unbound detecting-antibody (e.g., by washing) and then measuring the amount of the attached label using a detection method appropriate to the label. For example, where the label generates a color, the colored end product can represent the level or amount of cochlin antibodies. The level of detecting antibodies corresponds to the level of cochlin antibodies in the biological sample.

Once the amount level of the cochlin antibodies is determined, the amount can be compared to a predetermined value to provide information for determining if the subject potentially has or is at risk of age related and/or noise induced hearing loss. The predetermined value can be based upon the level of cochlin antibodies in comparable samples obtained from the general population or from a select population of subjects. For example, the select population can include apparently healthy subjects and/or subjects with ASNHL. “Apparently healthy”, as used herein, means subjects that show no indication of hearing loss (e.g., age related, noise induced hearing loss or ASNHL) or symptom of other hearing disorders.

The predetermined value can take a variety of forms. The predetermined value can be a single cut-off value, such as a median or mean. The predetermined value can be established based upon comparative groups such as where the level of cochlin antibodies in one defined group is double the level of the cochlin antibodies in another defined group. The predetermined value can be a range, for example, where the general population is divided equally (or unequally) into groups, or into quadrants, the lowest quadrant being subjects with the lowest levels of the cochlin antibodies, the highest quadrant being individuals with the highest levels of the cochlin antibodies.

The predetermined value can be derived by determining the level of antibodies in the general population. Alternatively, the predetermined value can be derived by determining the level of cochlin antibodies in a select population. For example, an apparently healthy population may have a different normal range of cochlin antibodies than a different ethnic or geographically located population based on the haplotype of such population. Accordingly, the predetermined values selected may take into account the category in which the subject falls. Appropriate ranges and categories can be selected with no more than routine experimentation by those of ordinary skill in the art.

Predetermined values of cochlin antibodies, such as for example, mean levels, median levels, or “cut-off” levels, are established by assaying a large sample of subjects in the general population or the select population and using a statistical model such as the predictive value method for selecting a positivity criterion or receiver operator characteristic curve that defines optimum specificity (highest true negative rate) and sensitivity (highest true positive rate).

Alternatively, the level of cochlin antibodies in the biological sample can be compared to a predetermined value to provide a risk value, which characterizes the subject's risk of developing age related and/or noise induced hearing loss.

The levels of cochlin antibodies in a biological sample from a subject may be compared to a single predetermined value or to a range of predetermined values. If the level of the present risk predictor in the test subject's bodily sample is greater than the predetermined value or range of predetermined values, the test subject is at greater risk of developing an age related and/or noise related hearing disorder than subjects with levels comparable to or below the predetermined value or predetermined range of values. In contrast, if the level of the present risk predictor in the test subject is below the predetermined value or range of predetermined values, the test subject is at a lower risk of developing an age related and/or noise induced hearing disorder than subjects with levels comparable to or above the predetermined value or range of predetermined values. For example, a test subject who has a higher level of a cochlin antibodies as compared to the predetermined value is at high risk of developing an age relate and/or noise induced hearing disorder, and a test subject who has a lower level of cochlin antibodies to the predetermined value is at low risk of developing an age related and/or noise induced hearing disorder. The extent of the difference between the test subject's risk predictor levels and predetermined value is also useful for characterizing the extent of the risk and thereby, determining which subjects would most greatly benefit from certain aggressive therapies. In those cases, wherein the predetermined value ranges are divided into a plurality of groups, such as the predetermined value ranges for subjects at high risk, average risk, and low risk, the comparison involves determining into which group the test subject's level of the relevant risk predictor falls. In an aspect of the invention, an increased level of cochlin antibodies in a biological sample obtained from the subject is indicative of age related and/or noise induced hearing loss in the subject.

The present invention further relates to a kit for assaying the presence of cochlin antibodies associated with age related and/or noise induced hearing loss in a subject. The kit includes cochlin protein, or a biologically active fragment thereof, and a solid support on which the cochlin can be immobilized. The kit can also include a detecting antibody that can be used to detect cochlin antibodies bound from biological samples contacted with the immobilized cochlin.

Diagnostic kits for the use in the present invention can be constructed by packaging the necessary materials, including cochlin, positive and negative controls, a solid support, and detecting antibody in a container with a set of instructions for performing the assay.

This invention is further illustrated by the following examples, which should not be construed as limiting.

### EXAMPLE

### Production of Recombinant Human Cochlin

Human cochlin cDNA generated as previously described (Robertson et al. (1997) Genomics 46:345-354), was inserted into pQE82L (Qiagen, Valencia, Calif.) for producing a His-tagged fused protein. XL1-Blue E. coli (Stratagene, La Jolla, Calif.) were transformed and screened for expression with HRP-conjugated His antibody (Qiagen). High level expression colonies were selected and the plasmid was maxiprepped and sequenced for verifying proper orientation and alignment. His-tagged cochlin was purified under denaturing conditions on a Ni-NTA agarose column (Qiagen). 10 μl of samples in denaturing SDS-PAGE buffer were loaded on a 15% Tris-HCL gel (Bio-Rad, Hercules, Calif.) and blotted onto Immobilon-P PVDF membrane (Millipore, Bedford, Mass.) and stained with HRP-conjugated His antibody (Qiagen). Detection was performed with the ECL Western Blotting Analysis system (Amersham Biosciences, Piscataway, N.J.) and exposure to Biomax MR film (Kodak, Rochester, N.Y.). Molecular weight was determined by Kaleidoscope Prestained Standards (Bio-Rad). Cochlin purity was optimized by HPLC purification of the Ni-NTA product using a Beckman System Gold 126 solvent module (Beckman Coulter, Fullerton, Calif.) with a Vydac C4 semi-preparative column (Grace Vydac, Hesperia, Calif.). Initial acetonitrile concentration was 5% and was ramped up in a linear fashion to 95% over 45 minutes. Detection was performed with a Beckman System Gold 166 detection module reading at 280 nm. Peak cochlin elution occurred at an acetonitrile concentration of 61%. Purified cochlin fractions were collected and lyophilized overnight on a Savant ModulyoD-115 system (Thermo Electron, Waltham, Mass.). The HPOC purified recombinant human cochlin was resuspended in double distilled deionized H₂O, and the final concentration was determined using a Bio-Rad Protein Assay (Bio-Rad, Hercules, Calif.).

### Selection of Study Subjects.

Main entry criteria for selection of the study subjects were those defined recently by Harris et al. (2003) JAMA 290:1875-1883. Inclusion criteria were defined as patients with bilateral sensorineural hearing loss of at least 30 decibels (dB), and progression of loss in at least one ear within three months as measured by a 10 dB pure tone worsening over three consecutive test frequencies and/or a 16% decrease in word discrimination score using a 25 word list. Exclusion criteria were defined as patients with congenital or genetic disease or patients with acquired otologic disease other than idiopathic or sudden hearing loss as well as patients with any additional autoimmune abnormalities as determined by clinical and medicinal history. Thus, the study subjects represented a patient population believed to have an organ-specific “primary” autoimmune disorder involving only inner ear abnormalities (Table I)

Although the study and a previously published national study (Harris et al. (2003)) did not restrict participation to any ethnic group(s), all patients in our study and 90% in the national study were Caucasian. The basis for this high Caucasian frequency is currently unclear. Normal hearing age- and sex-matched control study subjects were selected based on no prior clinical or medicinal history of either hearing loss or other immune abnormalities (Table I).

TABLE I
Patients and Age- and Sex-Matched Normal Hearing Control Study Subjects

| | | | Hearing | | | | | | | | | |
|---|---|---|---|---|---|---|---|---|---|---|---|---|
| | | | Loss^{B} | | Hearing Drop | | Disease | | | | | |
| ASNHL | Sex/ | 68 kD | (dB PTA) | | Within^{C} | | Duration^{D} | | Steroid Response^{E} | | Control | Sex/ |
| Patients | Age | Antibody^{A} | Right | Left | Right | Left | Right | Left | Right | Left | Subjects | Age |
| P1^{F} | F/73 | Negative | 48.8 | 33.8 | 3 mo. | 1 mo. | 4 yrs. | 2 mos. | Negative | Negative | C1 | F/73 |
| P2^{F} | F/69 | Negative | 52.5 | 65 | S.P. | 1 wk. | 19 yrs. | 2 mos. | Negative | Negative | C2 | F/69 |
| P3 | F/53 | Negative | 48.8 | 45 | 1 mo. | 3 mos. | 1 yr. | 2 yr. | + | + | C3 | F/52 |
| P4 | F/54 | Negative | 25 | 81 | 1 wk. | 1 wk. | 1 mo. | 5 yrs. | +++ | +++ | C4 | F54 |
| P5 | F/51 | Negative | 43.8 | 26.3 | 1 mo. | 1 mo. | 5 mos. | 1 yr. | ++ | +++ | C5 | F/51 |
| P6 | M/41 | Negative | 38 | N.R. | 2 mos. | S.P. | 6 yrs. | 39 yrs. | ++ | Negative | C6 | M/41 |
| P7 | F/57 | Negative | 60 | 22 | 3 mos. | 1 mo. | 15 yrs. | 1 mo. | Negative | +++ | C7 | F58 |
| P8 | F/49 | N.D. | 60 | 68 | 1 mo. | S.P. | 12 yrs. | 22 yrs. | + | + | C8 | F/40 |

^{A}68 KD antibody represents anti-HSP70. N.D. indicates not determined.
^{B}Hearing loss indicates the degree of each patient's dB hearing level determined as the pure tone average (PTA) of 500, 1000, and 2000 Hz at the time of ELISPOT testing. N.R Indicates no response.
^{C}Time over which hearing loss occurred in weeks (wk), months (mo), or slowly progressive (S.P.)
^{D}Disease duration indicates the period between onset of hearing loss and ELISPOT testing in weeks (wk), months (mo), or years (yr).
^{E}Degree of hearing improvement measured by dB PTA after steriod therapy: −none; + 10-20 dB increment; ++ 20-30 dB; +++ restored normal hearing.
^{F}2/8 (25%) of our ASNHL patients failed to show corticosteriod responsiveness.

Selection of Control Study Subjects with Other Hearing Loss (OHL) Abnormalities.

In addition to the proceeding subjects suspected of having ASNHL, we evaluated cochlin serum antibody titers in patients with bilateral sensorineural hearing loss not associated with rapidly progressive inner ear disease or with any immune or autoimmune disorder (Table II). These OHL control study subjects included patients with noise induced hearing loss and patients with presbycusis (age related hearing loss). Inclusion criteria were defined as patients with normal hearing in both ears early in life, recent gradual progressive hearing loss equally in both ears, >60 years of age for presbycusis, >40 years of age for noise induced hearing loss, normal otoscopic examination with audiogram showing bilateral symmetric sensorineural hearing loss unless mild asymmetry is due to noise exposure greater to one ear. Exclusion criteria were defined as patients with a family history of genetic hearing loss, ototoxic medication, corticosteroids taken in recent three months for any reason or for hearing loss at any time previously, rapid progression of hearing loss occurring within three months or less, history of systemic immune disease, prior or concomitant diagnosis of other ear disease, abnormal otoscopy, or audiogram showing conductive or mixed hearing loss in one or both ears or substantial asymmetry not explained by noise exposure greater to one ear.

TABLE II
Patients with Other Hearing Loss (OHL) Abnormalities.

| | | | Hearing | | Most Likely |
|---|---|---|---|---|---|
| | | History | Loss^{A} | | Cause of |
| Patient | Age/Sex | of Noise | (dB PTA) | WDS^{B} | Hearing Loss^{C} |
| OHL1 | 46/M | Yes | 12 | 96% | Noise |
| OHL2 | 53/F | Yes | 12 | 96% | Noise |
| OHL3 | 55/F | Yes | 20 | 100% | Noise |
| OHL4 | 56/M | Yes | 10 | 96% | Noise |
| OHL5 | 57/M | Yes | 20 | 96% | Noise |
| OHL6 | 62/M | Yes | 46 | 84% | Noise and Age |
| OHL7 | 67/M | Yes | NA^{D} | NA^{D} | Noise and Age |

^{A}Hearing loss indicates the degree of each patient's dB hearing level determined as the pure tone average (PTA) of 500, 1000, and 2000 Hz at the time of phlebotomy.
^{B}The word discrimination score (WDS) measures the percentage of words that can be correctly identified when presented at a comfortable loudness level. One PTA and one WDS score is given for each patient because a symmetric hearing loss equal in both ears was required for each control, i.e. the hearing impairment is the same for both ears.
^{C}Usually a PTA greater than 20 decibels is considered impaired in an adult. The reason three of our OHL controls have PTA less than 20 dB is because both noise and age affect primarily the higher frequencies above 2000 Hz; the subjective impairment would not be evident in the PTA measurement but might still bother the patient enough to come in for testing. There is no reporting convention that averages test results in only the higher frequencies above the speech range. The PTA is the only reporting convention although it sometimes includes 3000 Hz as well. Generally a WDS better than 80% is considered normal. Very often WDS is preserved despite high frequency hearing loss from noise, age or both. This explains the apparent normal WDS scores as well. Once the audiogram review shows the high-tone loss objectively, the diagnosis of noise induced loss is based solely on the patient history (i.e. military service, recreational noise, or work-related noise of significant duration and/or intensity).
^{D}NA means audiogram not available. A retrospective review of patient charts did not identify any record of audiogram and the diagnosis was based on written records of the audiologist and clinician.
^{E}Age related loss was based on a history that did not suggest an alternative cause of hearing loss and the patient's age was 60 or more.

### Cochlin Antibody Titers.

Sera from ASNH, OHL, and control subjects were tested by direct ELISA for cochlin antibody titers. Recombinant human cochlin was plated at 10 μg/ml on 96-wellNunc-immuno plates, MaxiSorp (Nalge Nunc, Naperville, Ill.), and sera were added in duplicate wells at dilutions ranging from 1/4 through 1/2048. Presence of bound antibody was determined using a peroxidase-goat anti-human IgG heavy and light chain (Zymed, South San Francisco, Calif.) followed by sequential treatment with ABTS substrate and H₂O₂ (Sigma, St. Louis, Mo.). PBS was used as a control substitute for the secondary detection antibody. The reaction was stopped after 30 minutes by adding SDS/DMF, and absorbance at 405 nm was measured using a Wallac 1420 VICTOR2 multilabel ELISA reader (Perkin-Elmer, Shelton, Conn.).

### Results

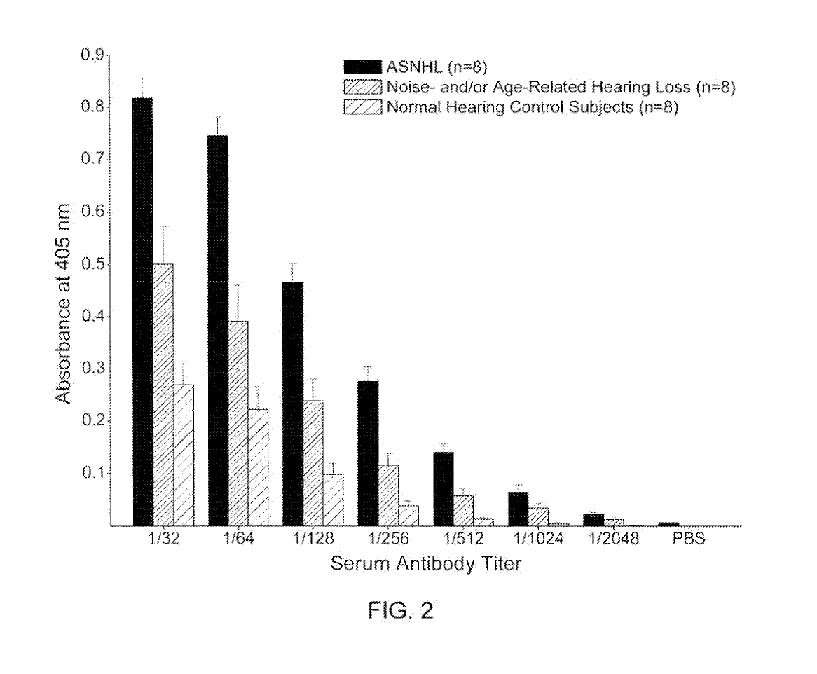

Sera from ASNHL patients, from patients with noise- and/or age related hearing loss (Table II), and from normal hearing age- and sex-matched control subjects were tested by direct ELISA for binding to recombinant human cochlin. At all serum dilutions from 1/32 through 1/2048, ASNHL patients showed significantly elevated titers to cochlin when compared to control OHL subjects with noise and/or age related hearing loss or to normal hearing control subjects (FIG. 2). Surprisingly, the cochlin serum antibody titers of OHL subjects were also significantly higher than those of age- and sex-matched controls. Differences in titers between all three study groups were significant at all dilutions tested from 1/32 to 1/2048 with P values ranging from P<0.05 to P<0.0005.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

Having described the invention, the following is claimed:

1. A method of detecting age related and/or noise induced hearing loss in a subject, the method comprising:
detecting in a biological sample from the subject the presence of cochlin antibodies; and
correlating the level of detected cochlin antibodies to the presence or absence of age related and/or noise induced hearing loss in the subject.

2. The method of claim 1, the detection of an increased level of cochlin antibodies in the biological sample compared to the level of cochlin antibodies in a control population of subjects that do not have age related and/or noise induced hearing loss indicates presence or increased risk of age related and/or noise induced hearing loss in the subject.

3. The method of claim 1, the cochlin antibodies being detected by an immunological method.

4. The method of claim 3, the immunological method comprising an ELISA.

5. The method of claim 1, detecting in the biological sample the presence of the cochlin antibodies comprising:
contacting the biological sample with cochlin;
contacting. cochlin antibodies bound to the cochlin with detectable antibodies; and
determining the level of the detectable antibodies bound to the cochlin antibodies, wherein the level of bound detectable antibodies corresponds to the level of cochlin antibodies in the biological sample.

6. The method of claim 5, the cochlin being immobilized on a solid support.

7. The method of claim 1, the biological sample comprising blood plasma, serum, or whole blood from the subject.

8. A method of detecting age related hearing loss in a subject, the method comprising:
detecting in a biological sample from the subject the presence of cochlin antibodies; and
correlating the level of detected cochlin antibodies to the presence or absence of age related hearing loss in the subject.

9. The method of claim 8, the detection of an increased level of cochlin antibodies in the biological sample compared to the level of cochlin antibodies in a control population of subjects that do not have age related indicates the presence or increased risk of age related hearing loss in the subject.

10. The method of claim 8, the cochlin antibodies being detected by an immunological method.

11. The method of claim 10, the immunological method comprising an ELISA.

12. The method of claim 8, detecting in the biological sample the presence of a cochlin antibodies comprising:
contacting the biological sample with cochlin;
contacting cochlin antibodies bound to the cochlin with detectable antibodies;
determining the level of the detectable antibodies bound to the cochlin antibodies, wherein the level of bound detectable antibodies corresponds to the level of cochlin antibodies in the biological sample.

13. The method of claim 12, the cochlin being immobilized on a solid support.

14. The method of claim 12, the biological sample comprising blood plasma, serum, or whole blood from the subject.

15. The method of claim 13, the cochlin immobilized on the solid support comprising recombinant human cochlin.

16. A method of detecting noise induced hearing loss in a subject, the method comprising:
detecting in a biological sample from the subject the presence of cochlin antibodies; and
correlating the level of detected cochlin antibodies to the presence or absence of noise induced hearing loss in the subject.

17. The method of claim 16, the detection of an increased level of cochlin antibodies in the biological sample compared to the level of cochlin antibodies in a control population of subjects that do not have noise related indicates the presence or increased risk of noise induced hearing loss in the subject.

18. The method of claim 16, the cochlin antibodies being detected by an immunological method.

19. The method of claim 18, the immunological method comprising an ELISA.

20. The method of claim 16, detecting in the biological sample the presence of a cochlin antibodies comprising:
contacting the biological sample with cochlin;
contacting cochlin antibodies bound to the cochlin with detectable antibodies;
determining the level of the detectable antibodies bound to the cochlin antibodies, wherein the level of bound detectable antibodies corresponds to the level of cochlin antibodies in the biological sample.

21. The method of claim 20, the cochlin being immobilized on a solid support.

22. The method of claim 16, the biological sample comprising blood plasma, serum, or whole blood from the subject.

23. The method of claim 21, the cochlin immobilized on the solid support comprising recombinant human cochlin.
